(19) United States
(12) Patent Application Publication
(10) Pub. No.: US 2005/0245909 A1
(43) Pub. Date: Nov. 3, 2005

(54) Embedded data chip in a surgical handpiece

(76) Inventors: Brian Douglas McCary, Clayton, MO (US); Allen W. Skinner, Florissant, MO (US)

Correspondence Address: Bausch & Lomb Incorporated, One Bausch & Lomb Place, Rochester, NY, 14604-2701, US

(21) Appl. No.: 10/835,034

(22) Filed: Apr. 29, 2004

(51) Int. Cl. A61B 17/00

(52) U.S. Cl. 606/1

(57) ABSTRACT

A surgical handpiece 10 includes an elongated housing 12 having a distal end 14 and a proximal end 16. The housing 12 is configured to receive a surgical instrument 18 at the distal end 14 and has a power cord 20 attached to the proximal end 16 for attachment to a surgical control console 34. An electronic data device 26 is fixed within the housing 12 for storing data related to the surgical handpiece 10. The data device 26 includes a connector 28 for electronically connecting the data device 26 to the console 34 or other equipment for retrieving stored data.

### BACKGROUND OF THE INVENTION

1. Field of the Invention

The present invention is related to surgical handpieces, and more specifically, to surgical handpieces having an electronic device, such as memory, embedded within the handpiece for storing, retrieving, and transmitting data related to the surgical handpiece.

2. Description of Related Art

Presently, surgical handpieces such as ophthalmic phacoemulsification handpieces each have certain performance characteristics that may be slightly different from one surgical handpiece to the other. In a phacoemulsification handpiece, such variations include handpiece impedance, resonant frequencies of a crystal stack, and fluidic flow resistance.

To accommodate such variances handpiece-to-handpiece, present day surgical systems typically require a handpiece to be calibrated to the control system prior to each surgery. This calibration can currently take on the order of 30 seconds. This amount of time in busy operating rooms such as ophthalmic surgery centers may result in unwanted delays in surgery. Therefore, it would be advantageous to provide for a surgical handpiece that does not require calibration by the user, but rather can be automatically recognized by the surgical console and immediately configured for proper operation of the surgical handpiece with the surgical console.

In addition, present surgical handpieces are returned for routine maintenance or for repair from unexpected failures. It would be desirable from a manufacturing and service view if a surgical handpiece could have data related to the fault conditions in existence upon failure transmitted with the surgical handpiece back to the manufacturer or repair center.

### BRIEF DESCRIPTION OF DRAWINGS

FIG. 1 is a perspective view of surgical instrument in accordance with the present invention; and

FIG. 2 is a modified block diagram showing a surgical handpiece in accordance with the present invention connected to a surgical console.

### DETAILED DESCRIPTION OF THE PREFERRED EMBODIMENT

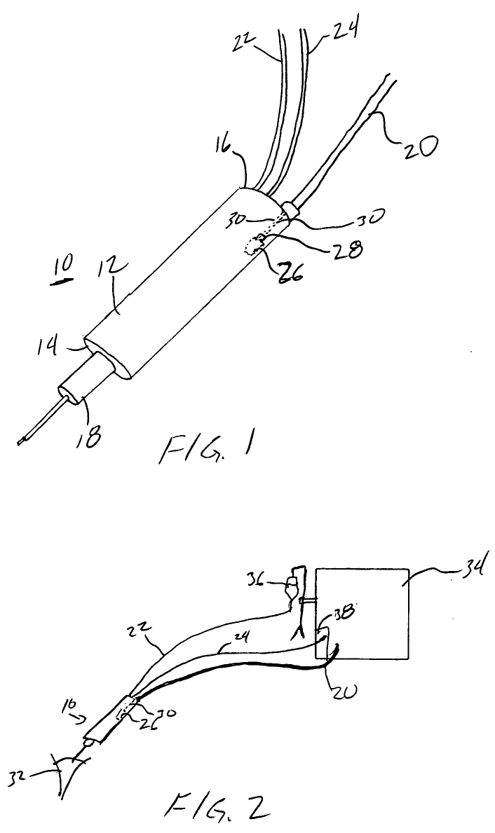

FIG. 1 shows a surgical handpiece 10, such as the phacoemulsification handpiece shown. However, it is to be appreciated that surgical handpiece 10 may be other handpieces than the phacoemulsification handpiece shown. Handpiece 10 may be an irrigation and aspiration handpiece, a laser probe, a vitrectomy cutter, a pneumatic scissors, or any other known surgical handpiece. Surgical handpiece 10 includes an elongated housing 12 having a distal end 14 and a proximal end 16. The housing 12 is configured to perform a surgical function, in this case phacoemulsification, or to receive a surgical instrument, such as the phaco needle 18 at the distal end 14. A power cord 20 is attached to the proximal end 16 for attachment to a surgical console (shown below in FIG. 2). It is to be understood that the power cord 20 could be attached such that the cord is attached to the side of proximal end 16, instead of as shown in FIG. 2. In addition, housing 12 may or may not have additional attachments, such as tubes 22 and 24, depending on the type of surgical handpiece.

An electronic data device 26, shown as a dashed box, is fixed within the housing 12 for storing data related to the surgical handpiece 10 and includes a connector 28 for electronically connecting the device 26 to the console or other equipment for retrieving the stored data. The device 26 is preferably electronically connected to the console via lines 30, shown as dash lines and incorporated into power cord 20.

The electronic data device 26 is preferably a non-volatile memory, such as EE PROM, flash memory, or any other suitable data storage device, which may be contained within housing 12 and which can withstand the rigors of autoclaving.

Device 26 may require two or three wires for proper operation. However, currently surgical handpieces typically include a shorting bar that provides evidence of a handpiece connection to the surgical console and uses two wires. The present invention can replace the shorting bar since the data device 26 will provide the necessary information for handpiece 10 to a control console and therefore, the two wires used for the shorting bar may be used for data device 26.

It is believed that data device 26 can provide many advantages to a user over the prior art handpieces. Such advantages include the storage of handpiece performance parameters, such as tuning parameters including impedance and resonant frequencies of operation of the handpiece 10. For example, in phacoemulsification handpieces, each handpiece 10 has a slightly different impedance and resonant frequency of its crystal oscillator stack. These differences presently require each handpiece to be calibrated upon each attachment to a console. This calibration can take on the order of 30 seconds to be accomplished. The present invention allows almost immediate calibration of the handpiece to a surgical console.

This immediate calibration is accomplished by storing performance parameters into device 26 during manufacture and also providing each device with a unique identifier. Upon connection of handpiece 10 to a console the performance parameters of that particular handpiece are transmitted via lines 30 in cord 20, to a surgical console, which then automatically and almost instantaneously configures itself for proper operation to that particular handpiece 10. Other performance parameters may include fluidic resistance and other parameters specific to the type of handpiece being used.

In addition, device 26 may allow for the storage of usage data. Such usage data may be downloaded from a surgical console to track the number of times a handpiece has been used. This information in turn can be used to warn a user that the handpiece needs to be serviced or its useful life is coming to an end.

Yet another advantage of the embedded data device 26 is that some pre-determined number of previous fault conditions associated with the handpiece could be downloaded from the console to the device 26. This could then help the manufacturer or service center determine the source of the fault or failure upon return from the user. This may also assist the manufacturer or service center in building a problem database in order to provide better service to the customer. In addition, the data device 26 may include a time and date stamp which could be used for warranty purposes or to identify the time and date of a fault occurrence. The time and date stamp can be useful for warranty issues by having a time and date of the sale embedded within the device 26.

Still another advantage of data device 26 is that customer profiling information may be recorded into the device 26 for each surgery performed. The customer profiling information may be many different pieces of data that include such information as the power consumption during use and the level of phaco power used for each pulse of phaco energy used. Similar information for other types of surgical handpieces may also be profiled and the data stored in data device 26.

FIG. 2 shows a modified block diagram of surgical handpiece 10 in use during surgery on an eye 32 and showing data device 26 connected via wires 30 and power cord 20 to a control console 34. FIG. 2 also shows tube 22 connected to irrigation source 36 and tube 24 connected to an aspiration pump 38. As can be seen, data device with non-volatile memory 26 is thereby electrically connected to the console 34. Data device 26 may also, via wires 30 and power cord 20, be connected to other equipment such as diagnostic equipment used at a service center.

1. A surgical handpiece comprising:
an elongated housing having a distal end;
the housing being configured to perform a surgical function or to receive a surgical instrument at the distal end and having a power cord attached to the housing for attachment to a surgical control console; and
an electronic data device fixed within the housing for storing data related to the surgical handpiece and including a connector for electronically connecting the electronic data device to the console or other equipment for retrieving the stored data.

2. The invention of claim 1, wherein the surgical handpiece is one taken from the group consisting of phacoemulsification, irrigation and aspiration, laser probe, vitrectomy cutter, and pneumatic scissors hand pieces.

3. The invention of claim 1, wherein the electronic data device is a non-volatile memory.

4. The invention of claim 1, where the connector includes one or more wires incorporated into the power cord.

5. The invention of claim 1, wherein the data stored includes handpiece performance parameters.

6. The invention of claim 1, wherein the data stored includes handpiece usage data.

7. The invention of claim 1, wherein the data stored includes handpiece customer profiling.

8. The invention of claim 1, wherein the data stored includes handpiece fault condition.

9. The invention of claim 1, wherein the data stored includes handpiece unique identifier.

10. The invention of claim 1, wherein the console or other equipment can cause additional data to be stored on the electronic data device.

11. A surgical handpiece comprising:
a housing having a distal end;
the housing being configured to perform a surgical function or to receive a surgical instrument at the distal end and having a power cord attached to the housing for attachment to a surgical console or other equipment; and
a non-volatile memory fixed within the housing for storing data, transmitting data, and receiving data related to the surgical handpiece, the memory including a connector for electronically connecting the memory to the console or other equipment.

12. The invention of claim 11, wherein the surgical handpiece is one taken from the group consisting of phacoemulsification, irrigation and aspiration, laser probe, vitrectomy cutter, and pneumatic scissors hand pieces.

13. The invention of claim 11, wherein the electronic data device is a non-volatile memory.

14. The invention of claim 11, where the connector includes on or more wires incorporated into the power cord.

15. The invention of claim 11, wherein the data stored includes handpiece performance parameters.

16. The invention of claim 11, wherein the data stored includes handpiece usage data.

17. The invention of claim 11, wherein the data stored includes handpiece customer profiling.

18. The invention of claim 11, wherein the data stored includes handpiece fault condition.

19. The invention of claim 11, wherein the data stored includes handpiece unique identifier.
